(19) United States
(12) Plant Patent Application Publication
(10) Pub. No.: US 2013/0340139 P1
(43) Pub. Date: Dec. 19, 2013

(54) Campanula plant named 'PKMP10'

(50) ;

(71) Applicant: Christian Hald Madsen, Korsor (DK)

(72) Inventor: Christian Hald Madsen, Korsor (DK)

(73) Assignee: Gartneriet PKM A/S

(21) Appl. No.: 13/507,227

(22) Filed: Jun. 14, 2012

(51) Int. Cl. A01H 5/00

(52) U.S. Cl. PLT/414

(57) ABSTRACT

A new and distinct cultivar of Campanula plant named ‘PKMP10’, characterized by its compact plant habit; globular plant form; vigorous growth habit; upright dark violet flowers and small leaves.

### LATIN NAME OF GENUS AND SPECIES OF THE PLANT CLAIMED

Campanula portenschlagiana Schult.

### VARIETY DENOMINATION

‘PKMP10’

### BACKGROUND OF THE INVENTION

The present invention relates to a new and distinct cultivar of Campanula plant, botanically known as Campanula portenschlagiana Schult., commonly known as Dalmatian Bellflower, and hereinafter referred to by the name ‘PKMP10’.

The new Campanula, ‘PKMP10’, is a product of a planned breeding program conducted by the inventor, Christian Hald Madsen, in Søhus, Denmark. The new Campanula originated from a cross made by the inventor in August 2009 between a proprietary selection of Campanula portenschlagiana Schult. designated 08.08.1001 (breeder reference, unpatented) as the female parent and a proprietary selection of Campanula portenschlagiana Schult. designated 08.08.1001 (breeder reference, unpatented) as the male parent. The new Campanula ‘PKMP10’ was discovered and selected by the inventor in August 2010 as a single flowering plant within the progeny of the stated cross in a controlled environment in Søhus, Denmark. The inventor selected ‘PKMP10’ on the basis of its compact and freely flowering plant habit.

Asexual reproduction of the new Campanula cultivar by terminal cuttings has demonstrated that the combination of characteristics as herein disclosed for the new cultivar are firmly fixed and retained through successive generations of asexual reproduction. The new cultivar reproduces true-to-type.

### SUMMARY OF THE INVENTION

The following traits have been repeatedly observed and are determined to be the unique characteristics of ‘PKMP10’. These characteristics in combination distinguish ‘PKMP10’ as a new and distinct cultivar:

1. Upright plant habit with short upright stems.

2. Bushy globular plant form.

3. Vigorous growth habit, but still needing less chemical growth retardation.

4. Small and dark violet flowers with wide and long petals.

5. Large quantity of buds.

6. No need for vernalization

Plants of the cultivar ‘PKMP10’ can be compared to the Campanula portenschlagiana Schult. cultivar ‘PKMP09’ (patented, U.S. Plant Pat. No. 22,774). In side-by-side comparisons conducted by the inventor in Stige, Denmark, plants of the instant cultivar ‘PKMP10’ and plants of the cultivar ‘PKMP09’ differ in the following characteristics:

1. Plants of ‘PKMP10’ have smaller buds and flowers than plants of ‘PKMP09’.

2. Plants of ‘PKMP10’ have a larger number of buds than ‘PKMP09’.

3. Plants of ‘PKMP10’ have a darker stem color than plants of ‘PKMP09’.

Plants of the cultivar ‘PKMP10’ can also be compared to the parent Campanula portenschlagiana Schult. cultivar 08.08.1001 (breeder reference, unpatented). Plants of the instant cultivar ‘PKMP10’ and plants of the parent cultivar 08.08.1001 differ in the following characteristics:

1. Plants of ‘PKMP10’ have darker flowers and buds than plants of ‘08.08.1001’.

2. Plants of ‘PKMP10’ are more upright and freely flowering than plants of ‘08.08.1001’.

### BRIEF DESCRIPTION OF THE PHOTOGRAPHS

The accompanying colored photographs illustrate the overall appearance of the new Campanula ‘PKMP10’, showing the colors as true as it is reasonably possible to obtain in colored reproductions of this type. Colors in the photographs may differ slightly from the color values cited in the detailed botanical description, which more accurately describe the actual colors of ‘PKMP10’.

FIG. 1 comprises a side perspective view of a typical flowering plant of PKMP10 grown in an 11 cm pot.

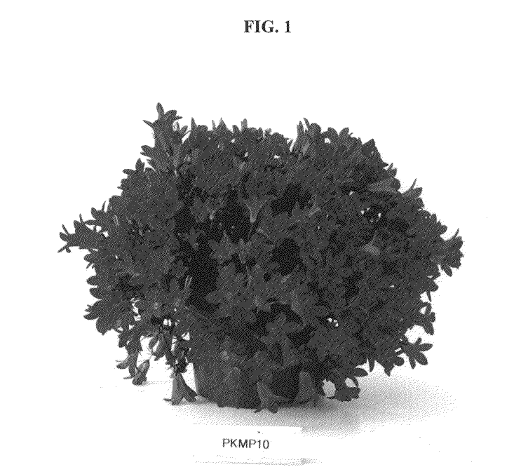

FIG. 2 comprises a close-up view of a typical young bud of PKMP10.

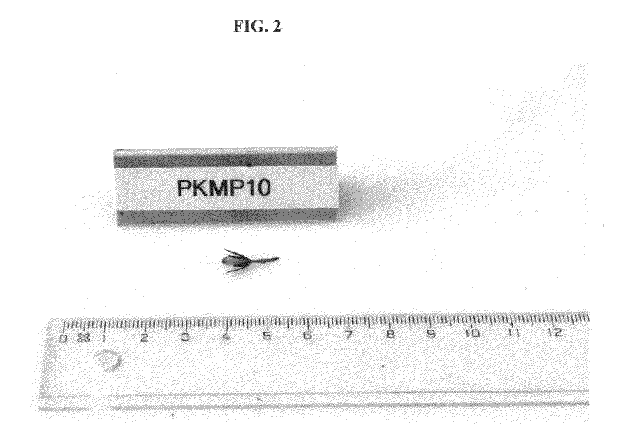

FIG. 3 comprises a close-up view of a typical mature bud of PKMP10.

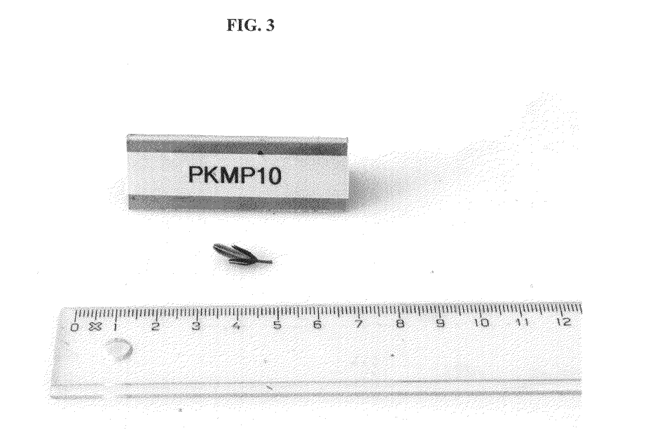

FIG. 4 comprises a close-up side perspective view of a typical flower of PKMP10.

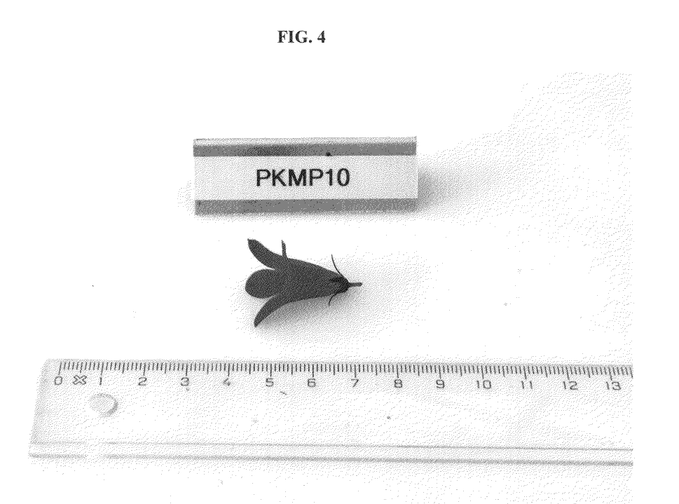

FIG. 5 comprises a close-up front perspective view of a typical flower of PKMP10.

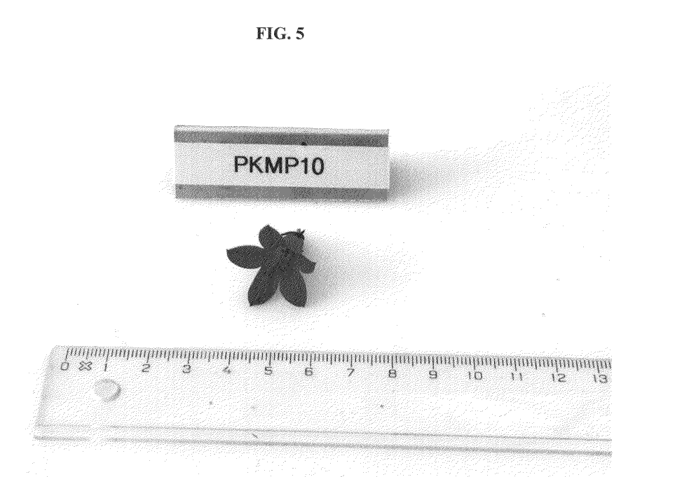

FIG. 6 comprises a close-up view of a typical basal leaf of PKMP10.

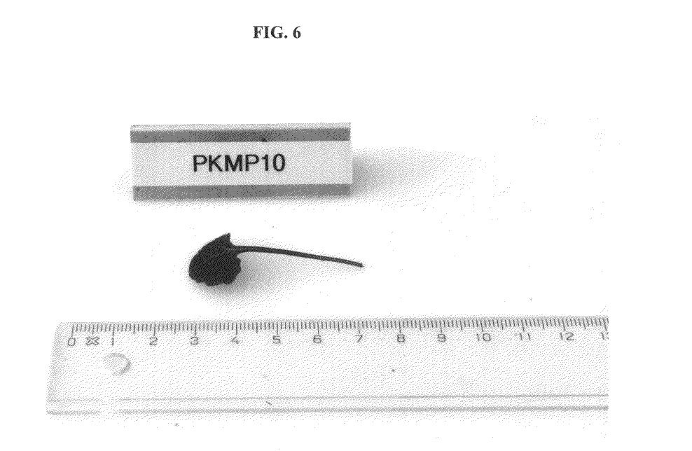

FIG. 7 comprises a close-up view of a typical apical leaf of PKMP10.

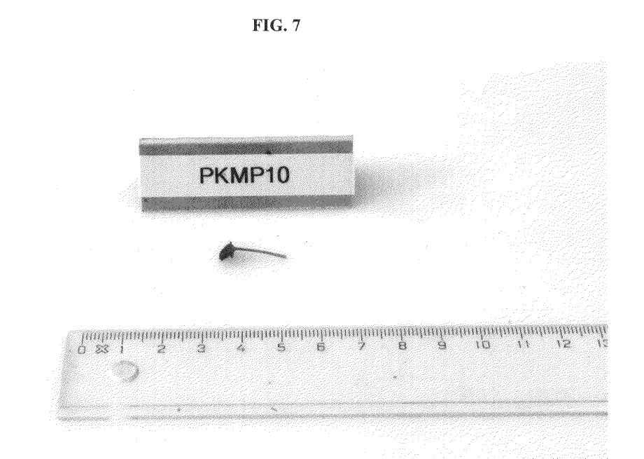

FIG. 8 is a comparison view of a typical flowering plant of PKMP10 with a typical flowering plant of PKMP09.

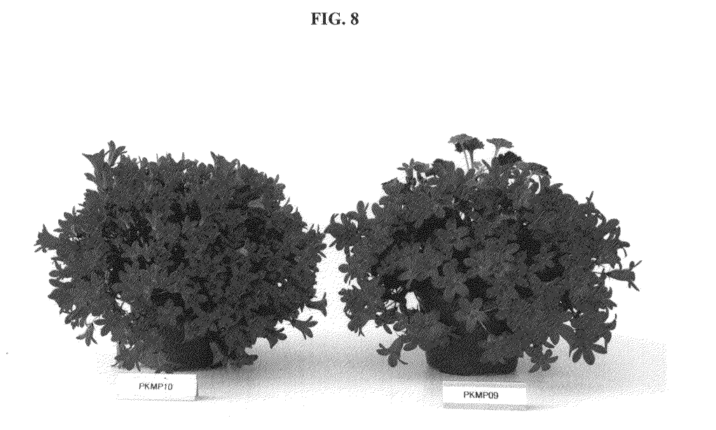

FIG. 9 is a comparison view of a typical flower of PKMP10 with a typical flower of PKMP09.

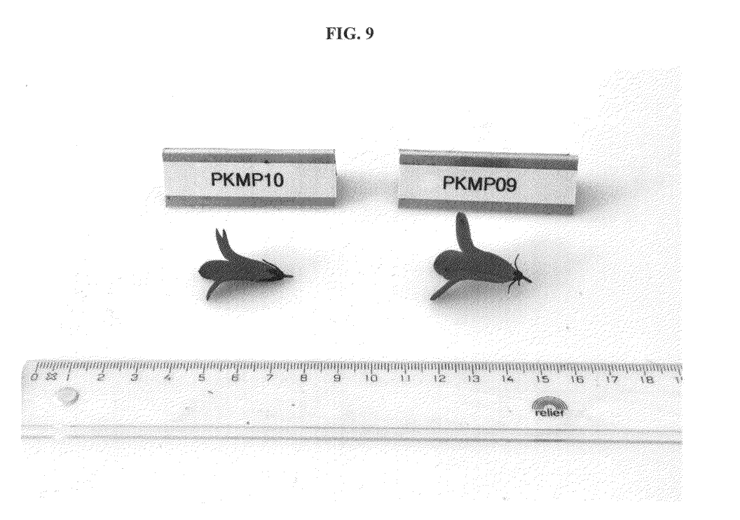

### DETAILED BOTANICAL DESCRIPTION

The new Campanula ‘PKMP10’ has not been observed under all possible environmental conditions. The phenotype of the new cultivar may vary with variations in environment such as temperature, light intensity, day length, and fertility level without any variance in genotype.

The aforementioned photographs, together with the following observations, measurements and values describe plants of the new Campanula ‘PKMP10’ as grown under greenhouse conditions. No growth retardants were used.

The age of the ‘PKMP10’ plants described is 10 months after cutting. Color references are made to The Royal Horticultural Society Colour Chart (R.H.S.), 5^{th} edition, except where general colors of ordinary significance are used.
Classification:
   Botanical.—Campanula portenschlagiana Schult.
Parentage:
   Female or seed parent.—Campanula portenschlagiana Schult. designated 08.08.1001 (breeder reference, unpatented).
   Male or pollen parent.—Campanula portenschlagiana Schult. designated 08.08.1001 (breeder reference, unpatented).
Propagation:
   Type cutting.—Terminal vegetative cuttings.
   Time to initiate roots.—About 10 to 14 days at 18 to 21° C. in tunnels in a greenhouse.
   Root description.—Fine, well branched.
Plant description:
   Form.—Perennial, rosette plant with upright plant habit. Campanulate flowers in racemes. Freely branching with lateral branches forming at every node; dense and bushy.
   Crop time.—After rooting, about 14 weeks are required to produce finished flowering plants in 11 cm pots.
   Plant height (from soil level to top of plant plane).—About 9 cm.
   Vigor.—Vigorous growth rate.
Foliage description:
   Leaf shape.—Single, dentate, cordate, palmate venation.
   Leaf length.—15-17 mm.
   Leaf width.—About 20-22 mm.
   Apex.—Rounded.
   Base.—Cordate.
   Margin.—Broadly dentate.
   Texture.—Smooth, glabrous, dull.
   Color.—Upper Surface, RHS N137A, Green Lower Surface, RHS N137A, Green.
   Venation color.—Upper Surface, RHS N137A, Green Lower Surface, RHS N137A, Green.
Inflorescence description:
   Flower arrangement and shape.—Single, upright, campanulate flowers in racemes, flowers with small star shaped calyx.
   Natural flowering season.—Continuous throughout spring and summer. Season can be extended through long day treatments, no vernalization needed.
   Flower longevity.—Longevity of individual flowers is highly dependent on temperature and light conditions. Flowers persistent.
   Inflorescence size.—Length: 5-7 cm.
   Flowers.—Length: About 20 mm Diameter: About 23 mm.
   Petals.—Overall Shape: Lanceolate, acute. Length: 8-10 mm Width: Base of lobe 6 mm. Corolla Color: Upper and Lower surfaces, RHS N87A, violet. The bottom of the bell has the same color.
   Sepals.—Arrangement: Shiny and free. Quantity per flower: 5. Length: 15 mm. Width: 5 mm. Overall shape: Hastate, sessile, clasping. Tip: Caudate. Base: Sessile. Margin: Entire. Texture: Pubescent, very short, stiff hairs. Color (mature and immature): Upper and lower surfaces: Green, RHS 138A. Peduncle: Strength: Moderately Strong Length: About 27 mm Diameter: About 1 mm Color: RHS 144A, yellow-green.
Reproductive organs:
   Gynoecium.—Pistil and Stigma: Shape: Conspicuous Tripartite Color: RHS N85A, violet.
Seed/fruit: None observed.
Weather tolerance: Plants of the new Campanula have exhibited good tolerance to drought, rain and wind, with low temperature resistance to −15° C.

I claim:

1. A new and distinct cultivar of Campanula plant named ‘PKMP10’, as illustrated and described herein.
